(19) United States
(12) Patent Application Publication
(10) Pub. No.: US 2025/0149149 A1
(43) Pub. Date: May 8, 2025

(54) ALIGNMENT OF DIGITAL REPRESENTATIONS OF PATIENT DENTITION

(71) Applicant: James R. Glidewell Dental Ceramics, Inc., Newport Beach, CA (US)

(72) Inventors: Fedor Chelnokov, Khimki (RU); Andrei Aralov, Maykopskaya (RU); Roman Skvortsov, Nerekhta (RU); Sergei Azernilov, Irvine, CA (US)

(73) Assignee: James R. Glidewell Dental Ceramics, Inc., Newport Beach, CA (US)

(21) Appl. No.: 18/501,546

(22) Filed: Nov. 3, 2023

(51) Int. Cl. G16H 30/40; G06T 7/00

(52) U.S. Cl. CPC G16H 30/40; G06T 7/0012; G06T 2207/30036

(57) ABSTRACT

A computer-implemented method/system/medium includes receiving a two dimensional (“2D”) digital image including at least a portion of a person's dentition; receiving a three dimensional (“3D”) digital surface model of the person's dentition; receiving one or more 3D digital surface model key points selected on the 3D digital surface model; receiving one or more corresponding 2D digital image key points selected on the 2D digital image; and fitting a camera model using the one or more 3D digital surface model key points and the one or more corresponding 2D digital image key points to align the 3D digital surface model with the 2D digital image.

### BACKGROUND

Dental treatment can benefit from utilizing multiple digital representations of a patent's dentition. One challenge in utilizing multiple digital representations can include displaying and working with the digital representations together. Manual solutions can include requiring a user control each rotation angle of a model and other parameters of the model and the camera and can be prone to inaccuracy and error. Aligning digital representations of patient dentition with one another can be challenging.

### SUMMARY

Disclosed is a computer-implemented method of aligning at least two digital representations of at least a portion of a patient's dentition. The computer-implemented method can include receiving a two dimensional (“2D”) digital image including at least a portion of a person's dentition; receiving a three dimensional (“3D”) digital surface model of the person's dentition; receiving one or more 3D digital surface model key points selected on the 3D digital surface model; receiving one or more corresponding 2D digital image key points selected on the 2D digital image; and fitting a camera model using the one or more 3D digital surface model key points and the one or more corresponding 2D digital image key points to align the 3D digital surface model with the 2D digital image.

Disclosed is a non-transitory computer readable medium storing executable computer program instructions to provide aligning at least two digital representations of at least a portion of a patient's dentition, the computer program instructions including instructions for: receiving a two dimensional (“2D”) digital image including at least a portion of a person's dentition; receiving a three dimensional (“3D”) digital surface model of the person's dentition; receiving one or more 3D digital surface model key points selected on the 3D digital surface model; receiving one or more corresponding 2D digital image key points selected on the 2D digital image; and fitting a camera model using the one or more 3D digital surface model key points and the one or more corresponding 2D digital image key points to align the 3D digital surface model with the 2D digital image.

A system for aligning at least two digital representations of at least a portion of a patient's dentition, the system including: a processor; and a non-transitory computer-readable storage medium including instructions executable by the processor to perform steps including: receiving a two dimensional (“2D”) digital image including at least a portion of a person's dentition; receiving a three dimensional (“3D”) digital surface model of the person's dentition; receiving one or more 3D digital surface model key points selected on the 3D digital surface model; receiving one or more corresponding 2D digital image key points selected on the 2D digital image; and fitting a camera model using the one or more 3D digital surface model key points and the one or more corresponding 2D digital image key points to align the 3D digital surface model with the 2D digital image.

### BRIEF DESCRIPTION OF THE DRAWINGS

FIG. 1 shows a Graphical User Interface (“GUI”) in some embodiments with a 3D digital surface model and a 2D digital image in some embodiments.

FIG. 2 shows a GUI in some embodiments with key points selected on a 3D digital surface model and a 2D digital image in some embodiments.

FIG. 3 shows a GUI with a control area in some embodiments.

FIG. 4(a) illustrates an example of a masked 2D digital image in some embodiments.

FIG. 4(b) illustrates of an example of a segmented 3D digital surface model in some embodiments.

FIG. 5(a) illustrates a masked 2D digital image in some embodiments.

FIG. 5(b) illustrates an example of determining the segmented 3D digital surface model centroids of the visible tooth region in terms of front view projected on the surface in the front direction.

FIG. 6 illustrates an example of an initial alignment/mapping after performing the first stage in some embodiments.

FIG. 7(a) illustrates an example of generating a masked digital surface image from the segmented 3D digital surface model in some embodiments.

FIG. 7(b) illustrates one example photographing in some embodiments.

FIG. 8 illustrates an example of contour pairs in some embodiments.

FIG. 9 illustrates a flowchart overview in some embodiments of automatically selecting points in some embodiments.

FIG. 10 illustrates an example in some embodiments of fitting a camera model in some embodiments.

FIG. 11 illustrates an example of fitting a camera in some embodiments.

FIG. 12 illustrates a flowchart of fitting a camera in some embodiments.

FIG. 13 illustrates a flowchart of the linear part of fitting a camera in some embodiments.

FIG. 14(a) illustrates a GUI in some embodiments of a mapped 2D image in some embodiments.

FIG. 14(b) illustrates an example in some embodiments of defining a cutout region.

FIG. 14(c) illustrates an example in some embodiments of a 3D digital surface model to which a mapped 2D digital image is mapped.

FIG. 15 illustrates a processing system in some embodiments.

### DETAILED DESCRIPTION

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the following description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

Some embodiments can include a computer-implemented method of aligning at least two digital representations of at least a portion of a patient's dentition. A digital representation can be a 2D digital image or a 3D digital surface model in some embodiments. The method can include, in some embodiments, displaying a Graphical User Interface (“GUI”) with one or more GUI elements to load a first and second digital representations corresponding to at least a portion of a patient's dentition, display the first and second digital representations, determine one or more key points from the first digital representation and one or more corresponding key points from the second digital representation, and perform alignment of the first digital representation with the second digital representation based on the one or more first digital representation key points and the corresponding one or more second digital representation key points. In some embodiments, the computer-implemented method can display the first digital representation and the second digital representation overlaid after their alignment. In some embodiments, the computer-implemented method can allow adjusting visibility of the aligned first digital representation and the second digital representation as they are displayed.

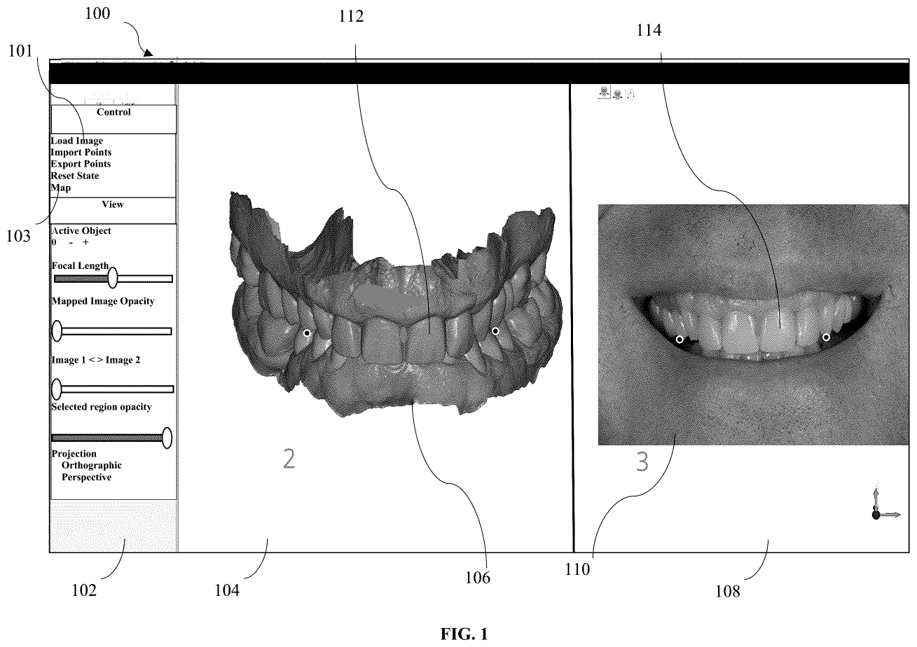

FIG. 1 illustrates one example of a GUI in some embodiments. GUI 100 can include one or more GUI regions, such as a first GUI region 102 to display one or more control and viewing GUI elements. For example, a GUI element such as load image button 101 can display a selection of files to load one or more digital representations of at least a portion of a patient's dentition. The GUI 100 can include a second area 104 to display a loaded first digital representation 106, and a third GUI region 108 to display a loaded second digital representation 110. Additional GUI control elements such as GUI sliders can adjust mapping features such as a mapping focal length and/or initiate alignment through a GUI button such as map button 103. Other GUI sliders can determine how the aligned and overlaid first and second digital representations are displayed. For example, GUI sliders can adjust a mapped (aligned) image opacity, image to image opacity, a selected region opacity, as well the projection (orthographic or perspective).

Some embodiments can include a computer-implemented method of aligning a three dimensional (3D) digital surface model of at least a portion of the patient's dentition with a two dimensional (2D) digital image of at least a portion of the patient's detention. For example, in some embodiments, the first digital representation of at least a portion of the patient's dentition can include a 3D digital surface model and the second digital representation of at least a portion of the patient's dentition can include a 2D digital image.

In some embodiments, the computer-implemented method can receive the 2D digital image of at least a portion of a patient's dentition by a user or automated process loading the 2D digital image from local or remote storage.

In some embodiments, the 2D digital image can be a digital photo. In some embodiments, the digital photo is generated by a digital camera. In some embodiments, the digital photo camera can be a smartphone. In some embodiments, the digital photo camera can be any digital camera known in the art. The digital photo is taken by anyone, such as a dentist, a patient (as a selfie) or anyone else and/or taken automatically. The digital photo can be taken anywhere, including but not limited to in or outside of dental laboratory, a dental office, or any other suitable location.

In some embodiments, the digital photo can include at least a portion of a patient's mouth region, with one or more teeth exposed. In some embodiments, the digital photo can be of a patient's face, including the eyes and mouth region, with one or more teeth exposed. In some embodiments, the digital photo can be of a patient smiling, thereby exposing one or more teeth. In some embodiments, the exposed teeth can be upper jaw teeth. In some embodiments, the exposed teeth can be lower jaw teeth. In some embodiments, the exposed teeth can include at least some upper jaw teeth and some lower jaw teeth. In some embodiments, one or more upper and/or lower gum regions can also be exposed.

In some embodiments, the 2D digital image can include at least one virtual jaw with one or more virtual teeth corresponding to the one or more exposed teeth. In some embodiments, the 2D digital image can include a virtual upper jaw with one or more upper jaw virtual teeth corresponding to the one or more exposed upper jaw teeth. In some embodiments, the 2D digital image can include a virtual lower jaw with one or more lower jaw virtual teeth corresponding to the one or more exposed lower jaw teeth.

In some embodiments, the computer-implemented method can receive the 3D digital surface model of at least a portion of a patient's dentition by a user or automated process loading the 3D digital surface model from local or remote storage of any type known in the art.

In some embodiments, the 3D digital surface model can include at least one virtual jaw with one or more virtual teeth corresponding to the one or more teeth. In some embodiments, the 3D digital surface model can include a virtual upper jaw with one or more upper jaw virtual teeth corresponding to the one or more upper jaw teeth. In some embodiments, the 3D digital surface model can include a virtual lower jaw with one or more lower jaw virtual teeth corresponding to the one or more lower jaw teeth.

In some embodiments, the 3D digital surface model can include a 3D mesh. In some embodiments, at least a portion of the 3D digital surface model can include the same patient dentition as at least a portion of the 2D digital image. For example, at least one or more virtual teeth in the 3D digital surface model can correspond to one or more virtual teeth in the 2D digital image.

In some embodiments, the 3D digital surface model is generated from a direct scan of the patient's dentition, which can include scanning one or more teeth and optionally one or more gum regions. In some embodiments, the scan can be performed using an optical scanner. This will typically take place, for example, in a dental office or clinic and be performed by a dentist or dental technician. Alternatively, this can be performed anywhere an intraoral scanner can be used. Any optical scanner known in the art that can generate a 3D digital surface model can be used. In some embodiments, the optical scanner can include an intra oral scanner. In some embodiments, the scan can be performed using an X-ray or CT scanner.

In some embodiments, the 3D digital surface model can be generated from a scan of a physical dental impression of a patient's dentition. Each virtual jaw with one or more virtual teeth model can be generated by scanning a physical impression using any scanning technique known in the art including, but not limited to, for example, optical scanning, CT scanning, etc.

A conventional scanner typically captures the shape of the physical impression/patient's dentition in 3 dimensions during a scan and digitizes the shape into a 3 dimensional digital model. The first virtual jaw with one or more virtual teeth model and the second virtual jaw with one or more virtual teeth model can each include multiple interconnected polygons in a topology that corresponds to the shape of the physical impression/patient's dentition. In some embodiments, the polygons can include two or more digital triangles. In some embodiments, the scanning process can produce STL, PLY, or CTM files, for example that can be suitable for use with a dental design software, such as FastDesign™ dental design software provided by Glidewell Laboratories of Newport Beach, Calif. One example of CT scanning is described in U.S. Patent Application No. Us20180132982A1 to Nikolskiy et al., which is hereby incorporated in its entirety by reference. In some embodiments, the physical impression can be scanned using an optical scanner. In some embodiments, the optical scanner can include an intraoral scanner. In some embodiments, the scan is performed using any imaging technique known in the art to generate 3D digital models. In some embodiments, the 3D digital surface model can include a point cloud. In some embodiments, the 3D digital surface model can be a segmented 3D digital surface model, with labeled individual segmented virtual teeth.

In some embodiments, the computer-implemented method can display the loaded 3D digital surface model and the loaded 2D digital image in the GUI.

FIG. 1 illustrates an example of a loaded 3D digital surface model 106 and a loaded 2D digital image 110. The loaded 3D digital surface model 106 can include at least a portion of a patient's dentition 112, including one or more virtual teeth. The loaded 2D digital image 110 can include at least a portion of the same patient's dentition 114, including one or virtual teeth corresponding to one or more virtual teeth in the loaded 3D digital surface model 106.

In some embodiments, the computer-implemented method can receive one or more 3D digital surface model key points selected on the 3D digital surface model. In some embodiments, the computer-implemented method can receive at least two 3D digital surface model key points. In some embodiments, the computer-implemented method can receive one or more 2D digital image key points selected on the 2D digital image. In some embodiments, the computer-implemented method can receive at least two 2D digital image key points. In some embodiments, the one or more 3D digital surface model key points correspond to the one or more 2D digital image model key points. In some embodiments, the correspondence can be based on a corresponding location on a corresponding virtual tooth in each respective digital representation. In some embodiments, the corresponding 2D digital image key points and the 3D digital surface model key points can each form a pair of 2D-3D points. In some embodiments, the 2D digital image key points and the 3D digital surface model key points are selected manually by a user, for example. In some embodiments, the 2D digital image key points and the 3D digital surface model key points can be selected automatically. In some embodiments, the computer-implemented method can fit the entire set of 2D-3D points a camera (a.k.a. solving PnP), which can output a transform matrix and image position in the camera's coordinate system. The computer-implemented method can use the transform matrix and image position to arrange the 2D digital image in alignment with the 3D digital surface model.

### Manual Selection

The one or more 3D digital surface model key points and the one or more 2D digital image key points can be selected manually in some embodiments. In some embodiments, the one or more 3D digital surface model key points selected in the 3D digital surface model correspond to the same location as the one or more 2D digital image key points selected in the 2D digital image.

In some embodiments, the selection can be performed using the GUI that displays the 3D digital surface model and the 2D digital image. For example, a user such as a dentist, dental technician, or any other user can select the key points on the displayed 3D digital surface model and the displayed 2D digital image. The GUI can display the 3D digital surface model and the 2D digital image so that both are visible to the user at the same time, such as next to each other in some embodiments. However, other arrangements are possible.

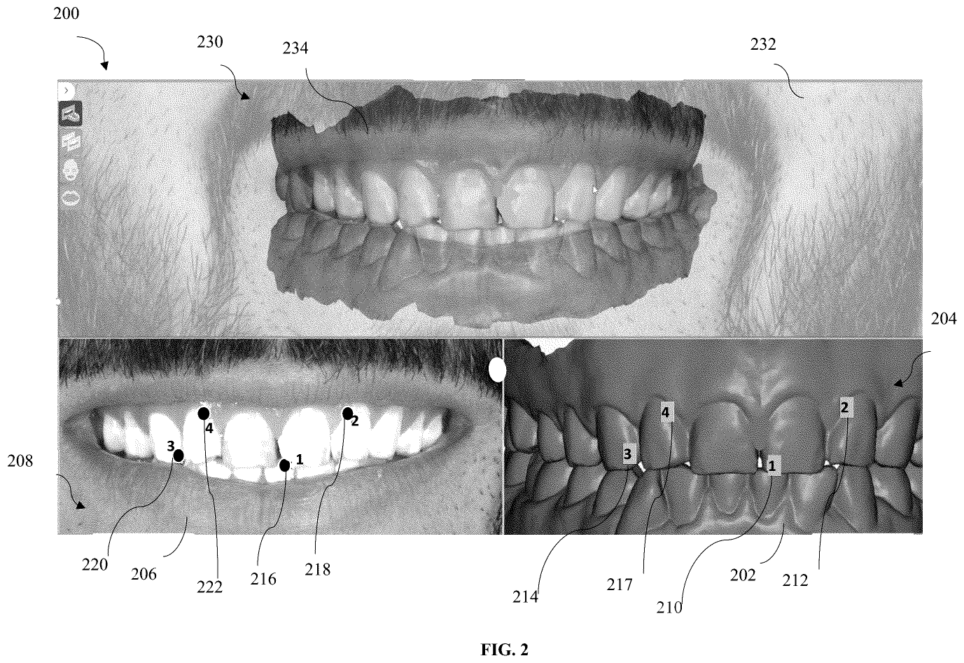

FIG. 2 shows a GUI 200 in some embodiments displaying the 3D digital surface model 202 in a first GUI section 204 and the 2D digital image 206 in a second GUI section 208 side by side. The GUI can allow viewing adjustments to the 3D digital surface model and/or the 2D digital image. For example, the viewing adjustments to the 3D digital surface model and/or the 2D digital image can include rotating, moving, scrolling, zooming in and out, and/or any other common GUI manipulation known in the art. A user can select 3D digital surface model key points such as a first 3D digital surface model key point 210, a second 3D digital surface model key point 212, a third 3D digital surface model key point 214, and a fourth 3D digital surface model key point 217 in the 3D digital surface model 204 by clicking on the location using an input device such as a mouse, stylus, finger, or any other input device known in the art. As the user clicks on a location in the 3D digital surface model, the computer-implemented method can record the 3D coordinates of the location and can store the sequence number and the 3D coordinates of the selected location as a 3D digital surface model key point and display the 3D digital surface model key point in the 3D digital surface model at the selected location along with its sequence number in some embodiments. In the example of FIG. 2, the computer-implemented method can store the sequence number and 3D coordinates of the first, second, third, and fourth 3D digital surface model key points. In some embodiments, clicking on an already selected 3D digital surface model key point deletes the key point from the 3D digital surface model. More or fewer points can be selected; the number shown in the figure is for illustrative purposes.

Similarly, a user can select 2D digital image key points such as a first 2D digital image key point 216, a second 2D digital image key point 218, a third 2D digital image key point 220, and a fourth 2D digital image key point 222 in the 2D digital image 208 by clicking on the location using an input device such as a mouse, stylus, finger, or any other input device known in the art. As the user clicks on a location in the 2D digital image, the computer-implemented method can record the 2D coordinates of the location and can store the sequence number and the 2D coordinates of the selected location as a 2D digital image key point and display the 2D digital image key point in the 2D digital image at the selected location along with its sequence number in some embodiments. In the example of FIG. 2, the computer-implemented method can store the sequence number and 3D coordinates of the first, second, third, and fourth 2D digital image key points. In some embodiments, clicking on an already selected 2D digital image key point deletes the key point from the 2D digital image. More or fewer points can be selected; the number shown in the figure is for illustrative purposes.

In some embodiments, a key point in the 3D digital surface image corresponds to a key point in the 2D digital image in location, and vice versa. For example the first 3D digital surface model key point 210 is in the same corresponding location on the corresponding virtual tooth in the 3D digital surface model 202 as the first 2D digital image key point 216 is on in the 2D digital image 206. In some embodiments, the computer-implemented method can accordingly pair corresponding key points in the 3D digital surface model and the 2D digital image.

In some embodiments, the computer-implemented method can fit a camera model (solving the PnP problem) using the manually selected one or more 3D digital surface key points and the manually selected one or more 2D digital image key points as described in the Fitting A Camera Model section of the present disclosure to perform alignment/mapping.

In some embodiments, the computer-implemented method can use the resulting transformation matrix and position of the 2D digital image in a camera coordinate system from Fitting A Camera Model to place the 3D digital surface model and the 2D digital image (masked) relative to the camera and display both in the GUI.

In some embodiments, the computer-implemented method can display an overlay or mask of the aligned/mapped 2D digital image over the 3D digital surface model. For example, in FIG. 2, GUI section 230 can display the 2D digital image 232 masked over the 3D digital surface model 234.

Some embodiments of the computer-implemented method can include the GUI displaying a control area. In some embodiments, the control area can allow adjustments to a focal length. In some embodiments, the focal length can be adjusted for less than 5 pairs of 3D digital surface model key point and 2D digital image key points. In some embodiments, the control area allows adjustments to a mapped image opacity. In some embodiments, the image opacity can control the opacity of the mapped/aligned/masked 2D digital image with respect to the 3D digital surface model displayed.

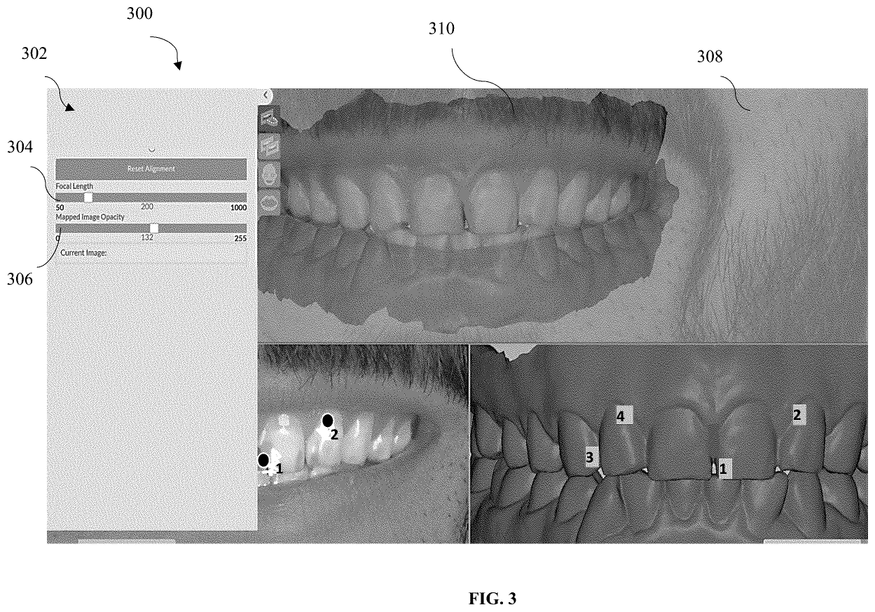

FIG. 3 shows an example of a GUI 300 in some embodiments. The GUI 300 can include a control area such as control area 302 that can allow adjustment of a focal length through a focal length slider 304 (or any other GUI element) and adjustment of the mapped 2D digital image opacity through a mapped image opacity slider 306 (or any other GUI element) in some embodiments. The control area 302 can be always visible or hidden until accessed via a GUI element such as a button to pop up, slide in or drop down to be visible in the GUI 300. In some embodiments, adjustments to the focal length and/or the mapped image opacity are applied directly to the mapped 2D digital image 308. For example the mapped 2D digital image can be more or less visible with respect to the 3D digital surface model as the are both displayed in a mapped and overlaid 2D digital image 310. This can allow, for example, visualization of the 3D digital surface model (and any modifications to it) with respect to the patient's photographed dentition.

### Automatic Selection and Mapping

In some embodiments, the computer-implemented method can perform alignment of the 2D digital image and the 3D digital surface model by automatically selecting the 2D digital image key points and the 3D digital surface model key points and performing alignment/mapping. In some embodiments, the computer-implemented method can perform alignment/mapping using automatic selection in two stages.

In some embodiments, the one or more segmented 3D digital surface model key points and the one or more 2D digital image key points can be selected automatically.

In some embodiments, automatic selection can include receiving a masked 2D digital image. The masked 2D digital image can include one or more masked individual virtual teeth each having a virtual tooth boundary, which can be visible. In a preferred embodiment, the masked 2D digital image can include three or more masked individual virtual teeth each having a virtual tooth boundary, which can be visible. In some embodiments, each masked individual virtual tooth in the masked 2D digital image can include a unique identifier or color. The masked 2D digital image can include masks of one or more virtual teeth in the virtual upper jaw and/or the virtual lower jaw of the 2D digital image. In some embodiments, the virtual teeth in the virtual upper jaw and/or virtual lower jaw are exposed, or not covered by virtual lips. In some embodiments, the masked 2D digital image can include a visible boundary for each masked virtual tooth.

The 2D digital image can be received pre-masked, prior to mapping/alignment using any tooth masking technique known in the art in some embodiments. For example, in some embodiments, the masked 2D digital image can be generated by a user defining each virtual tooth in the 2D digital image using an input device such as a mouse, finger, etc, for example, in the 2D digital image. Alternatively, in some embodiments, the masked 2D digital image can be generated as described in: “Evaluating the Precision of Automatic Segmentation of Teeth, Gingiva and Facial Landmarks for 2D Digital Smile Design Using Real-Time Instance Segmentation Network”, Seulgi Lee and Jong-Eun Kim, Journal of Clinical Medicine 11.3 (2022), the entirety of which is hereby incorporated by reference. As another alternative, in some embodiments, the masked 2D digital image can be generated as described in “Automated integration of facial and intra-oral images of anterior teeth”, Mengxun Li et al., Computers in Biology and Medicine 122, (2020), p. 103794, the entirety of which is hereby incorporated by reference.

In some embodiments, automatic selection can include receiving a segmented 3D digital surface model. In some embodiments, the segmented 3D digital surface model can include one or more segmented 3D virtual teeth, each can include a unique identifier or color. In a preferred embodiment, the segmented 3D digital surface model can include three or more segmented 3D virtual teeth, each can include a unique identifier or color. The segmented 3D digital surface model can be generated using any tooth segmentation technique known in the art. In some embodiments, the segmented 3D digital surface model can be segmented into individual virtual teeth and virtual gum. For example, in some embodiments, the segmented 3D digital surface model can be generated using one or more techniques/features described in TEETH SEGMENTATION USING NEURAL NETWORKS, U.S. patent application Ser. No. 17/140,739, filed Jan. 4, 2021, the entirety of which is hereby incorporated by reference. In some embodiments, the segmented 3D digital surface model can be generated using one or more techniques/features described in SEMIAUTOMATIC TOOTH SEGMENTATION, U.S. patent application Ser. No. 16/778,406, filed Jan. 31, 2020 the entirety of which is hereby incorporated by reference.

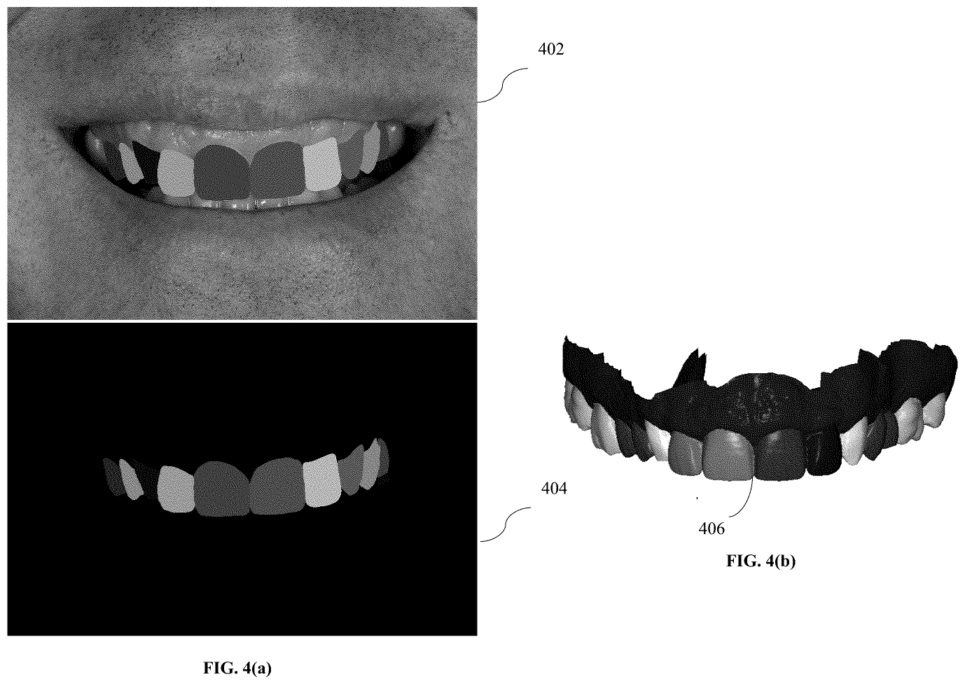

FIG. 4(a) illustrates an example of a masked 2D digital image 402 that can be received by the computer-implemented method. Also shown is the masked 2D digital image with virtual teeth only 404. One or more individual virtual teeth in the masked 2D digital image can each be uniquely identifiable. FIG. 4(b) illustrates an example of a segmented 3D digital surface model 406 that can be received by the computer-implemented method.

In some embodiments, automatic selection can include two stages. In some embodiments, in a first stage, the computer-implemented method can determine one or more initial 2D digital image key points and one or more initial 3D digital surface model key points. In some embodiments, the one or more initial 2D digital image key points and the one or more initial 3D digital surface model key points can include centroids of one or more virtual teeth in the 2D digital image and the 3D digital surface model.

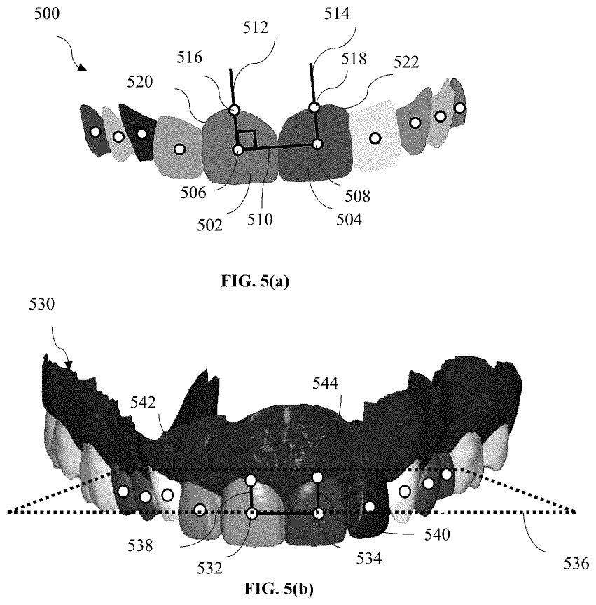

In some embodiments, determining initial 2D digital image key points in a first stage can include determining 2D digital image centroids of the one or more masked virtual teeth in the masked 2D digital image. The number of masked virtual teeth used from the masked 2D digital image can be at least four in some embodiments. FIG. 5(a) illustrates a masked 2D digital image 500 depicting ten virtual teeth masked so each individual tooth can be identified. The computer-implemented method has determined a centroid for each individual masked tooth in the masked 2D digital image 500 (depicted as circles in the center of each virtual tooth).

In some embodiments, determining the initial 2D digital image key points in a first stage can include determining two masked virtual teeth with known IDs (or, in some embodiments, dental types). In some embodiments, virtual teeth with maximum area can be selected (with known or unknown ID or/and dental type) for the first stage. In some embodiments, known frontal incisors can be selected. FIG. 5(a) depicts a first virtual central incisor 502 and a second virtual central incisor 504 as each having the largest area (or first and second-largest area) determined by the computer-implemented method for the masked 2D digital image 5(a).

In some embodiments, determining the initial 2D digital image key points in a first stage can include connecting a first masked virtual tooth centroid in the masked 2D digital image with a second masked virtual tooth centroid in the masked 2D digital image to provide a virtual centroid vector. In some embodiments, the first masked virtual tooth and second masked virtual tooth can be those with the maximum area. In some embodiments, for example, the first masked virtual tooth and the second masked virtual tooth can be frontal incisors. In some embodiments, connecting the first masked virtual tooth and the second masked virtual tooth can include determining a first masked virtual tooth normal as perpendicular to the virtual centroid vector and extending from the first masked virtual tooth centroid in the masked 2D digital model. The computer-implemented method can determine a second masked virtual tooth normal as perpendicular to the virtual centroid vector and extending from the from the virtual tooth centroid in the masked 2D digital image in some embodiments.

FIG. 5(a) illustrates determining the initial 2D digital image key points in a first stage in some embodiments. The computer-implemented method can connect a first virtual central incisor centroid 506 with a second virtual central incisor centroid 508 to provide the virtual centroid vector 510. The computer-implemented method can determine a first virtual incisor normal 512 as perpendicular to the virtual centroid vector 510 and extending from the first virtual central incisor centroid 506 in some embodiments. The computer-implemented method can determine a second virtual incisor normal 514 as perpendicular to the virtual centroid vector 510 and extending from the second virtual central incisor centroid 508 in some embodiments. Although virtual central incisors are shown in the example, other virtual teeth can be used in some embodiments.

The computer-implemented method can determine a first masked virtual tooth intersection as an intersection between the first masked virtual tooth normal and a first masked virtual tooth boundary in the masked 2D digital image in some embodiments. The computer-implemented method can determine a second masked virtual tooth intersection as an intersection between the second masked virtual tooth normal and a second masked virtual tooth boundary in the masked 2D digital image in some embodiments.

As illustrated in FIG. 5(a), the computer-implemented method can determine a first virtual incisor intersection 516 as an intersection between the first virtual incisor normal 512 and a first masked virtual incisor boundary 520. The computer-implemented method can determine a second virtual incisor intersection 518 as an intersection between the first virtual incisor normal 514 and a first masked virtual incisor boundary 522.

In some embodiments, automatic selection can include determining one or more initial segmented 3D digital model surface key points in a first stage. In some embodiments, determining the one or more initial segmented 3D digital surface model key points can include determining segmented 3D digital surface model centroids of one or more virtual teeth in the segmented 3D digital surface model. In some embodiments, the 3D digital surface model centroids are of the corresponding virtual teeth in the masked 2D digital image.

In some embodiments, determining the segmented 3D digital surface model centroids in a first stage can include using an orthogonal camera mode to view the virtual jaw in the segmented 3D digital surface model from a front position. The computer implemented method can determine all segmented 3D digital surface model centroids of visible vertices for each virtual tooth in the segmented 3D digital surface model and project the determined segmented 3D digital surface model centroids onto a surface of a corresponding virtual tooth in the direction of view in the segmented 3D digital surface model.

FIG. 5(b) illustrates an example of determining the segmented 3D digital surface model centroids in a first stage in some embodiments. The computer-implemented method can arrange a segmented 3D digital surface model 530 using an orthogonal camera mode to view the virtual jaw from the front position. The computer-implemented method can determine one or more segmented 3D digital surface model centroids such as a first virtual incisor centroid 532 and second virtual incisor centroid 534. Although virtual central incisors are shown in the example, other virtual teeth can be used in some embodiments.

In some embodiments, determining one or more initial segmented 3D digital model surface key points can include determining a first masked virtual tooth normal as perpendicular to a plane perpendicular to a screen and extending from the first virtual central incisor centroid in the masked 2D digital model. In some embodiments, perpendicular to the screen can include a direction that produces a portrait view looking at the virtual jaw from a front direction. In some embodiments, the screen can include a plane generated by taking a cross product between a vector in the portrait direction with a vector connecting the centroids of the virtual central incisors. In some embodiments, the screen can include a plane perpendicular to the portrait view direction. In some embodiments, determining one or more initial segmented 3D digital model surface key points can include determining a second masked virtual tooth normal as perpendicular to the plane perpendicular to a screen and extending from the from the virtual central incisor centroid in the segmented 3D digital surface model.

FIG. 5(b) illustrates a plane 536 perpendicular to the portrait view direction. The computer-implemented method can determine first virtual incisor normal 538 as perpendicular to the plane 536 and second virtual incisor normal 540 as perpendicular to the plane 536.

In some embodiments, the computer-implemented method can determine a first masked virtual tooth intersection as an intersection between the first masked virtual tooth normal and a first masked virtual tooth boundary in the segmented 3D digital surface model. In some embodiments, the computer-implemented method can determine a second masked virtual tooth intersection as an intersection between the second masked virtual tooth normal and a second masked virtual tooth boundary in the segmented 3D digital surface model.

As illustrated in FIG. 5(b), the computer-implemented method can determine a first virtual incisor intersection 542 and second virtual incisor intersection 544.

In some embodiments, the one or more initial segmented 3D digital model surface key points can correspond to the one or more initial 2D digital image surface key points for the corresponding virtual tooth.

In some embodiments, the computer-implemented method can fit a camera model as described in the Fitting A Camera Model section by providing one or more pairs of initial 2D image key points and corresponding initial segmented 3D digital model key points. Fitting a camera model can return a transform matrix and image coordinates (position) in the camera's coordinate system based on the initial 2D digital image surface key point and 3D digital surface key point pairs. In some embodiments, the initial camera model fit provides an initial transform matrix and initial image coordinates in (u,v) coordinate system to provide an initial map between the camera coordinate system of the 2D digital image and the world coordinate system of the segmented 3D digital surface model.

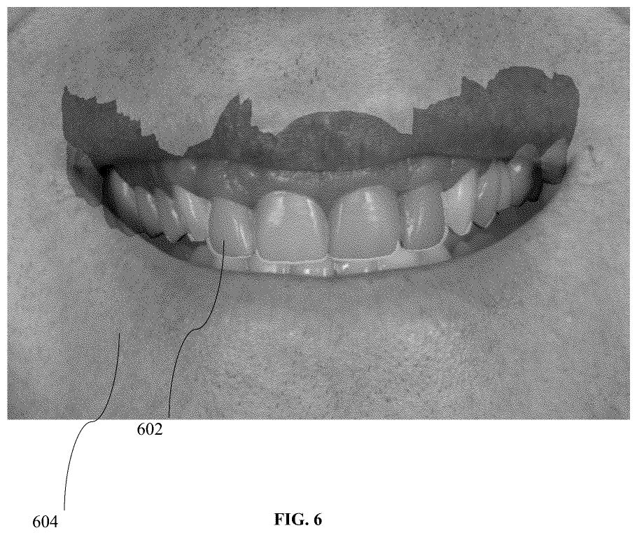

FIG. 6 illustrates an example of an initial alignment/mapping after performing the first stage. As can be seen, the 3D digital surface model 602 and the initially mapped 2D digital image align 604, and the 3D digital surface model can be seen with the virtual teeth in the initially mapped 2D digital image. In some embodiments, the computer-implemented method can take the results of fitting the camera model from the first stage and perform a second stage to further align the 2D digital image with the 3D digital surface model.

### Second Stage

In some embodiments, performing the second stage can include generating a masked digital surface image from the segmented 3D digital surface model based on an input transform matrix and image coordinates of the 2D digital image. In an initial iteration of the second stage, the input transform matrix can be the initial transform matrix and the image coordinates can be initial image coordinates. In each subsequent iteration of the second stage, the input transform matrix can be the output transform matrix from the previous iteration of the second stage, and the input image coordinates can be output image coordinates from the previous iteration of the second stage.

In some embodiments, generating the masked digital surface image can include virtually photographing the segmented 3D digital surface model with a virtual camera in a perspective mode. In some embodiments, photographing can include arranging the virtual camera and a digital surface plane (“digital screen”) in the world coordinate system relative to the segmented 3D digital surface model using a current camera model fit. In some embodiments, the segmented 3D digital surface model is arranged between a digital surface plane and a virtual camera based on the current camera model. In some embodiments, the digital surface plane is positioned behind the model using the current transformation matrix. In some embodiments, the position and dimensions of the digital surface plane and the pixel size correspond to the original masked 2D digital image and In some embodiments, the digital surface plane's distance along the Z-axis of the camera to the camera itself. In some embodiments, the digital surface plane can include the same resolution as the masked 2D digital image of the photograph. In some embodiments, the digital surface plane is arranged orthogonal to the perspective view from the virtual camera. In some embodiments, the digital surface plane is positioned behind the segmented 3D digital surface model using the transformation matrix from the current camera model fit. In some embodiments, the position, dimensions, and pixel size of the digital surface plane will correspond to the masked 2D digital image and its distance along the Z-axis of the camera to the camera itself from the current camera model fit.

In some embodiments, photographing can include ray tracing. In some embodiments, the number of rays can include the resolution of the masked 2D digital image. In some embodiments, the number of rays can include the number of pixels in the masked 2D digital image. In some embodiments, the number of rays can include the number 3D digital surface model points intersected. In some embodiments, ray tracing can include projecting a ray from the virtual camera to a center of each pixel in the digital surface plane. In some embodiments, faces of the segmented 3D digital surface model mesh not facing the virtual camera are ignored.

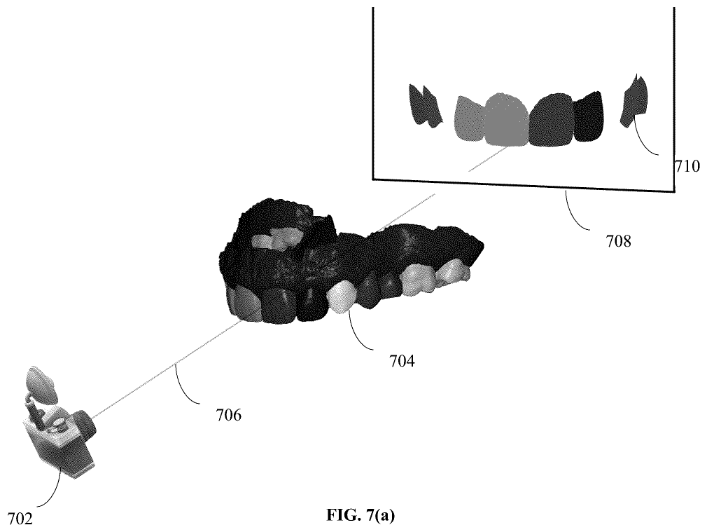

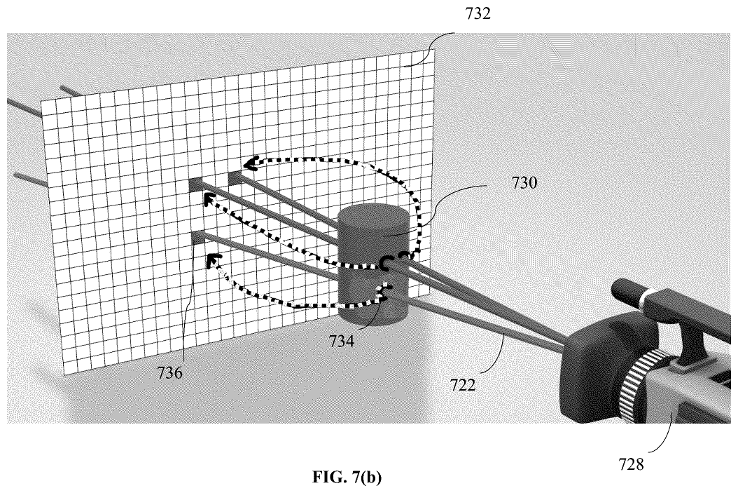

FIG. 7(a) illustrates an example of generating a masked digital surface image from the segmented 3D digital surface model by virtually photographing in some embodiments. In some embodiments, the segmented 3D digital surface model 704 is arranged between the virtual camera 702 and the digital surface plane 708. A virtual camera 702 in a perspective view with the 3D digital surface model 704 can utilize rays such as ray 706 onto a digital surface plane 708 to generated the masked digital surface image 710. The masked digital surface image can be a 2D version of the 3D digital surface model after virtually photographing.

In some embodiments, each ray intersecting the segmented 3D digital surface model is recorded in the digital surface plane. In some embodiments, ray tracing correlates a point on the segmented 3D digital surface model with a pixel in the digital surface plane. In some embodiments, ray tracing provides a point mapping between the 3D digital surface model and the 2D digital surface plane. In some embodiments, for each ray intersecting the segmented 3D digital surface model, recording in a corresponding pixel in the digital surface plane a color of the surface where the particular ray intersects the segmented 3D digital surface model for the first time. In some embodiments, each intersecting ray maps 3D coordinates of a ray intersection on the segmented 3D digital surface model to 2D coordinates of the corresponding pixel in the digital surface plane. In some embodiments, mapping can include recording 2D coordinates of the corresponding pixel for each ray along with the corresponding 3D coordinates of where the particular ray for the pixel intersects the segmented 3D digital surface model for the first time. In some embodiments, the masked digital surface image can include one or more individual virtual surface image teeth each having a unique color or identifier corresponding to its virtual tooth in the segmented 3D digital surface model.

FIG. 7(b) illustrates one example photographing in some embodiments. One or more rays such as ray 722 can be projected from a virtual camera 728 in perspective mode through the segmented 3D digital surface model 730 and onto the digital surface plane 732. As ray 722 passes through the segmented 3D digital surface model 730 at a first segmented 3D digital surface model location 734, the computer-implemented method records the color (or virtual tooth identifier) to which the first segmented 3D digital surface model location 734 belongs in a first digital surface plane pixel 736 where the first ray 722 intersects the digital surface plane. The computer-implemented method can map the first segmented 3D digital surface model location 734 with the first digital surface plane pixel 736.

In some embodiments, the computer-implemented method can correlate one or more contour points on the masked digital surface image with one or more contour points in the masked 2D digital image. In some embodiments, the one or more contour points on the masked digital surface image can form a closed boundary for a virtual tooth in the masked digital surface image. In some embodiments, the one or more contour points on the masked 2D digital image can form a boundary for a virtual tooth in the masked 2D digital image. In some embodiments, a contour point is determined as a virtual tooth pixel having at least one neighboring pixel with a different color or identifier. In some embodiments, since the masked digital surface image and the masked 2D digital image coincide in position, resolution, and size, correlating can include determining for each 1^{st}, 2^{nd}, . . . N contour point on the masked digital surface, a geometrically closest point of all contour points in the masked 2D digital image. In some embodiments, geometrically closest can include Euclidean distance in pixel (integer) coordinates. In some embodiments, the one or more points in the masked 2D digital image can form a contour of one or more virtual teeth in the masked 2D digital surface image.

In some embodiments, the contours are of one or more virtual teeth for a selected virtual jaw. In some embodiments, the one or more virtual teeth are not covered by lips or teeth of an unselected virtual jaw. In some embodiments, the selected virtual jaw is a top virtual jaw. In some embodiments, the selected virtual jaw is a bottom virtual jaw.

In some embodiments, the computer-implemented method can pair one or more masked 2D digital image contour points with one or more corresponding segmented 3D digital surface model contour points through the masked digital surface image. The masked digital surface image can therefore link masked 2D digital image contour points with corresponding segmented 3D digital surface model contour points. Each link can constitute a 2D digital image contour point/3D digital surface model contour point pair.

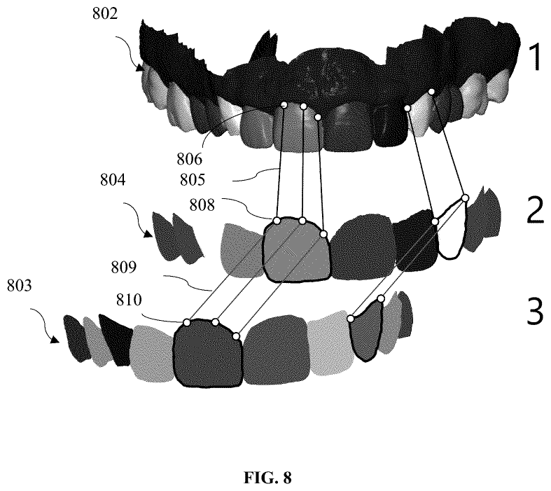

FIG. 8 illustrates an example of contour pairs in some embodiments. The figure shows a segmented 3D digital surface model 802 and a masked digital surface image 804 generated by the computer-implemented method by photographing the segmented 3D digital surface model 802. Also shown in the example is a masked 2D digital image 803. In the example, the computer-implemented method maps 805 a first segmented 3D digital surface model contour point 806 to a first masked digital surface image contour point 808 by photographing the segmented 3D digital surface model 802. In some embodiments, the computer-implemented method correlates 809 a first masked 2D digital image contour point 810 with the first masked digital surface image contour point 808. The masked digital surface image 804 thus links the first masked 2D digital image contour point 810 and the first 3D digital surface model contour point 806.

In some embodiments, the computer-implemented method can fit a camera model using the 2D digital image contour point/3D digital surface model contour point pairs as described in the Fitting a Camera Model section of the present disclosure. In some embodiments, the camera model fit provides a transform matrix and image coordinates in (u,v) coordinate system.

In some embodiments, the computer-implemented method can perform additional iterations of the second stage. In some embodiments, the computer-implemented method can perform each iteration of the second stage using the results from the previous iteration of the second stage as input. For example, in some embodiments, the computer-implemented method can use a transform matrix and image position output from the previous iteration of the second stage as input to the current iteration of the second stage to determine and use contours and 2D digital image point/3D digital surface model point pairs corresponding to the contours as described in the second stage. In some embodiments, the computer-implemented method can fit a camera model for the current iteration of the second stage. In some embodiments, each iteration of the second stage can provide a more accurate transform matrix and image coordinates. In some embodiments, the second stage can be repeated until a user-configurable iteration counter is reached or a condition of sufficient quality of the solution. In some embodiments, the number of iterations can be 10 or less.

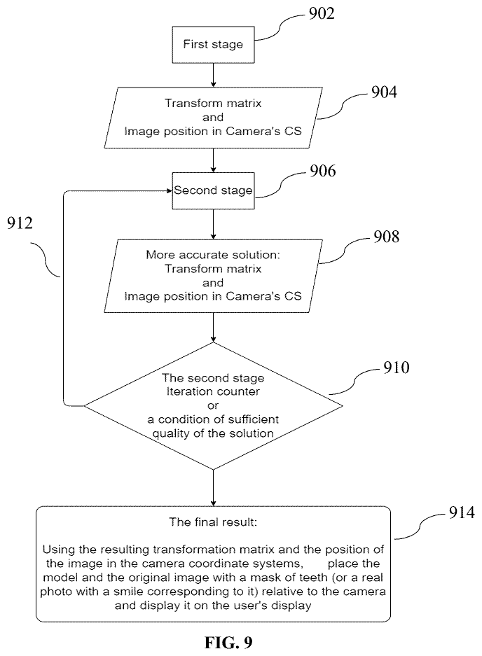

FIG. 9 illustrates a flowchart overview in some embodiments of automatic arrangement of points. The computer-implemented method can perform the first stage at 902. The first stage output at 902 can include a transform matrix and image position in the camera's coordinate system at 904. The computer-implemented method can perform the second stage 906 using the first stage output 902 as input. The second stage output 908 can include a more accurate transform matrix and image position the camera's coordinate system. The computer-implemented method can determine at 910 whether the second stage iteration counter has reached its user configurable value or if a condition of sufficient/desired quality of the solution is reached. If not, the computer-implemented method can loop 912 to perform additional iterations of the second stage 906, using as input the more accurate transform matrix and image position in the camera's coordinate system from the immediately prior iteration. If so, then the computer can, using the resulting transformation matrix and the position of the image in the camera coordinate system, place the model and the original image with a mask of virtual teeth (or a real photo with a smile corresponding to it) relative to the camera and display it on the user's display 914.

In some embodiments, the computer-implemented method can display the automatically selected key points in the 2D digital image (or masked 2D digital image) and in the 3D digital surface model (or segmented 3D digital surface model). In some embodiments, the computer-implemented method can allow a user to optionally manually delete or manually select automatically chosen key points in the 2D digital image (masked 2D digital image) and/or the 3D digital surface model (segmented 3D digital surface model).

In some embodiments, a final output can include placing the 3D digital surface model and the masked 2D digital image (or real photo with corresponding smile) relative to the camera using the final transformation matrix and displaying both on the user's display. In some embodiments, the computer-implemented method can provide an opacity GUI control to adjust the opacity of the 3D digital surface model relative to the 2D digital image.

### Fitting A Camera Model

In some embodiments, the computer-implemented method can receive one or more 3D digital surface model points and their corresponding 2D digital image points and fit a camera model. Each 3D digital surface point and its corresponding 2D digital image point can be referred to as a pair. In some embodiments, the 3D digital surface image points can be from a segmented digital surface model. In some embodiments, the 2D digital image points can be from a masked 2D digital image. They are referred to in this section as 3D digital surface model points and 2D digital image points. In some embodiments, the 3D digital surface image points and 2D digital image points can be selected manually as described in the “Manual selection” section of the present disclosure, as not only automatically selected points could be used to fit camera model.

In some embodiments, the 3D digital surface model key points are in a coordinate system of the world (model). In some embodiments, the 2D digital image key points are in a coordinate system of the 2D digital image. In some embodiments, the 2D digital image key points are in a plane perpendicular to a Z-axis of a camera. In some embodiments, the computer-implemented method can obtain a location of one or more 2D digital image key points in the camera coordinate system by setting a focal distance can include a distance to the 2D digital image plane.

In some embodiments, the camera coordinate system can include a 2D digital image origin point. In some embodiments, the distance between two or more 2D digital image key points can be measured in pixels. In some embodiments, the distance between two or more 2D digital image key points can be measured in dimensionless units.

In some embodiments, fitting can include determining a transformation matrix (“transform matrix”). In some embodiments, the transform matrix can include a rotation and a translation. In some embodiments, the transform matrix is from the world coordinate system to the camera coordinate system. In some embodiments, the transform matrix provides a location of all corners of the 2D digital image (u,v). In some embodiments, the transform matrix provides camera coordinates. In some embodiments, the location of all corners of the 2D digital image (u,v) and the camera coordinates can include an output of fitting the camera model. In some embodiments, the transform matrix maps the segmented 3D digital surface model key points to the 2D digital image key points. In some embodiments, fitting can include performing a transform of the 2D digital image key points and the segmented 3D digital surface model key points. In some embodiments, the fitting is performed automatically.

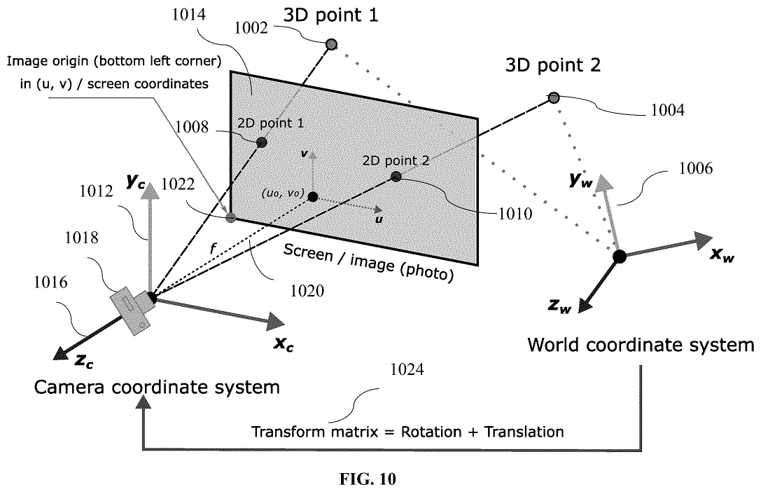

FIG. 10 illustrates an example in some embodiments of fitting a camera model. The 3D digital surface model key points first 3D point 1002 and second 3D point 1004 are in a coordinate system of the world (model) 1006. The 2D digital image key points first 2D point 1008 and second 2D point 1010 are in a camera coordinate system 1012 of the 2D digital image. In some embodiments, the 2D digital image key points are in a 2D digital image plane (screen) 1014 perpendicular to a Z-axis 1016 of a camera 1018. In some embodiments, the computer-implemented method can obtain a location of one or more 2D digital image key points in the camera coordinate system by setting a focal distance 1020 can include a distance to the 2D digital image plane 1014.

In some embodiments, the camera coordinate system can include a 2D digital image origin point 1022. In some embodiments, the distance between two or more 2D digital image key points can be measured in pixels. In some embodiments, the distance between two or more 2D digital image key points can be measured in dimensionless units.

In some embodiments, fitting can include determining a transformation matrix (“transform matrix”) 1024. In some embodiments, the transform matrix can include a rotation and a translation. In some embodiments, the transform matrix is from the world coordinate system 1006 to the camera coordinate system 1012. In some embodiments, the transform matrix provides a location of all corners of the 2D digital image (u,v). In some embodiments, the fitting is performed automatically.

In some embodiments, an algorithm for finding the best perspective transformation (transform matrix) given a set of pairs of points can be referenced as a PnP problem, for instance.

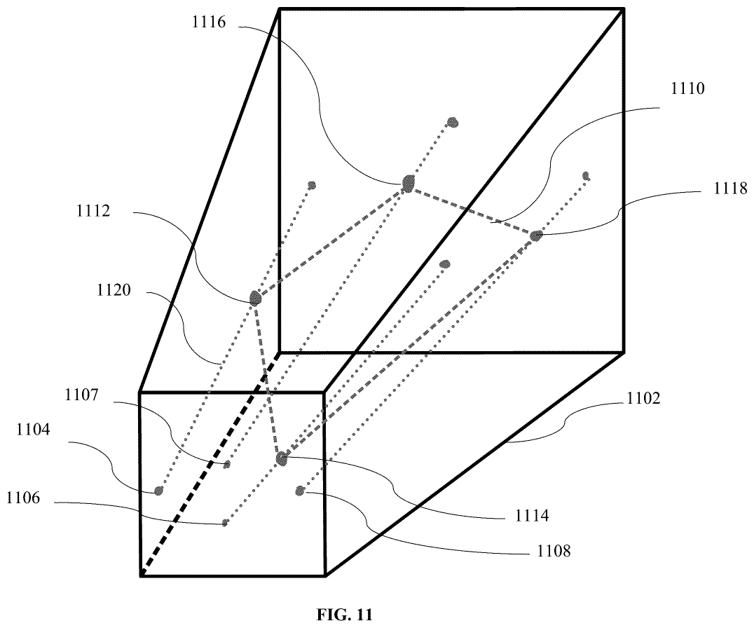

One visualization of this problem is demonstrated in the example shown in FIG. 11. FIG. 11 includes a camera frustum 1102. In the example, selected are one or more selected 2D digital image points such as first selected 2D digital image point 1104, second selected 2D digital image point 1106, third selected digital image point 1107, and fourth selected digital image point 1108. Also shown in the example is a 3D digital surface model 1110 with first selected 3D digital surface model point 1112, second selected 3D digital surface model point 1114, third selected 3D digital surface model point 1116, and fourth selected 3D digital surface model point 1118. Ray 1120 shows the trajectory to the virtual camera. The fact that ray 1120 originating from first selected 2D digital image point 1104 go through the first 3D digital surface model point 1112 means that the first selected 2D digital image point 1104 and the first 3D digital surface model point 1112 appear aligned on a screen.

Given the notation:

$$S = \{ ( p , q ) \vert p ∈ {R}^{3} , q ∈ {R}^{2} \} - set of points \\ T = ( \begin{matrix}{t}_{11} & {t}_{12} & {t}_{13} & {s}_{1} \\ {t}_{21} & {t}_{22} & {t}_{23} & {s}_{2} \\ {t}_{31} & {t}_{32} & {t}_{33} & {s}_{3} \\ 0 & 0 & 0 & 1\end{matrix} ) - perspective transformation matrix .$$

θ∈R³—image transformation
xₖ∈R^{k} for x∈Rⁿ denotes vector constructed from first k coordinates of vector x.

Given that, the objective is, in some embodiments:

$$F = \frac{1}{2} {\sum }_{p , q ∈ S} {‖ \frac{{( T \cdot p )}_{2}}{{( T \cdot p )}_{z}} - \frac{q + {θ}_{2}}{{θ}_{z}} ‖}^{2} \to {min}_{T , θ}$$

(the expression T·p implies using homogeneous coordinates, i.e. multiplying T·(pₓ p_{y} p_{z} 1)^{t})

In some embodiments, this is a nonlinear optimization problem, which has no direct analytic solution. In some embodiments, a more complicated approach involving two stages is used: (1) iterative linear approximation and (2) refinement with a gradient descent. Moreover, depending on the number of pairs |S| some parameters may or may not be optimized.

In some embodiments, a parametrization can be specified first more precisely. A first-order approximation of rotation part in the transformation matrix can be used, though other parametrizations could be used as well, including second-order approximation or direct usage of trigonometric functions in some embodiments.

$$T \approx ( \begin{matrix}1 & - γ & β & {s}_{1} \\ γ & 1 & - α & {s}_{2} \\ - β & α & 1 & {s}_{3} \\ 0 & 0 & 0 & 1\end{matrix} )$$

From this it follows that there are totally 9 parameters: θₓ, θ_{y}, θ_{z}, α, β, γ, s₁, s₂, s₃. In some embodiments, there can be 2 different cases: first, where the number of pairs of points is greater than or equal to 5, and second, where the number of pairs of points is between 2 and 5.

Number of pairs of points is greater or equal to 5. In this case all the model parameters can be optimized.

In some embodiments, the following can be performed where the number of pairs of points is greater or equal to 5.

In some embodiments, the following set of focuses may be used:

$$\{ {( \frac{{f}_{max}}{{f}_{min}} )}^{\frac{i}{m - 1}} \cdot {f}_{min} \vert i = \overline{1 … m - 1} \} ,$$

with varying fₘᵢₙ, fₘₐₓ, m. In some embodiments, a gradient descent-based method can be used to find the best focus according to the objective function, starting from different focuses from the aforementioned set.

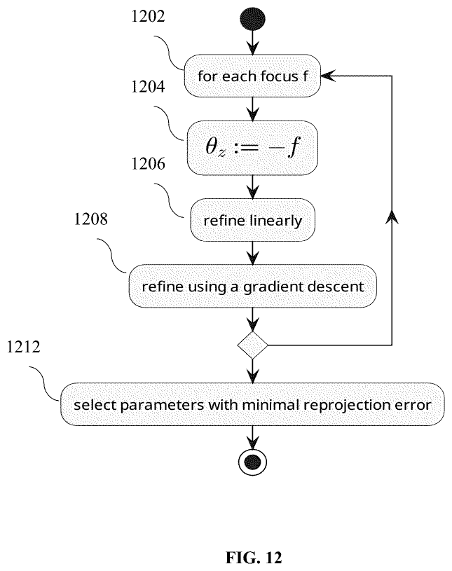

As illustrated in FIG. 12, for each focus at 1202 in the aforementioned set, use linear approximation, assuming θ_{z}=−f at 1204. Rewrite nonlinear system 1206 based on the objective. It has 2·|S| equations:

$$\{ \begin{matrix}\frac{{( Tp )}_{x}}{{( Tp )}_{z}} = \frac{{q}_{x} + {θ}_{x}}{{θ}_{z}} \\ \frac{{( Tp )}_{y}}{{( Tp )}_{z}} = \frac{{q}_{y} + {θ}_{y}}{{θ}_{z}} \\ …\end{matrix}$$

Transforming it so that there is no division terms and throwing out quadratic (with respect to parameters) terms gives linear system:

$$\{ \begin{matrix}{p}_{z} {θ}_{x} + 0 {θ}_{y} - {θ}_{z} {s}_{1} + 0 {s}_{2} + {q}_{x} {s}_{3} + {q}_{x} {p}_{y} α - ( {p}_{x} {q}_{x} + {θ}_{z} {p}_{z} ) β + {θ}_{z} {p}_{y} γ = 0 \\ 0 {θ}_{x} + {p}_{z} {θ}_{y} - 0 {s}_{1} - {θ}_{z} {s}_{2} + {q}_{y} {s}_{3} + ( {q}_{y} {p}_{y} + {p}_{z} {θ}_{z} ) α - {p}_{x} {q}_{y} β - {θ}_{z} {p}_{x} γ = 0 \\ …\end{matrix}$$

Since there are more than 5 pairs, the system has more than 10 pairs, thus it is overdetermined and could be solved via least squares method in some embodiments.

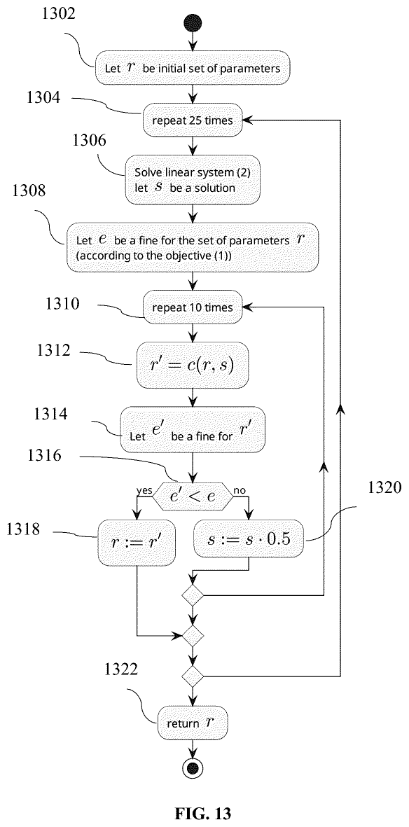

After that, find the appropriate weight of the solution and merge it with the current approximation. This could be done via an iterative process which stops if error decreases and multiplies each parameter by some coefficient less than 1 otherwise. One example of such a process in some embodiments is illustrated in FIG. 13.

Previous step is repeated until system converges or for a fixed number of iterations in some embodiments. This is also demonstrated in FIG. 13.

As illustrated in FIG. 13, the following steps can be performed: Let r be the initial set of parameters at 1302. The linear system can be solved at 1306, providing a solution s. Let e be a fine for the set of parameters r at 1308. r′=c(r,s) at 1312. Let e′ be a fine for r′ at 1314. If e′<e at 1316, then r:=r′ at 1318. Otherwise, s:=s·0.5 at 1320. In some embodiments, the computer-implemented method can loop back to step 1310 and repeat steps 1312, 1314, 1316 and 1318 or 1320 several times in an inner loop. In some embodiments, the computer-implemented method can repeat the inner loop 10 times. In some embodiments, the computer implemented method can, in an outer loop, repeat steps 1306, 1308, 1310, 1312, 1314, 1316, and 1318 or 1320 several times. In some embodiments, the computer-implemented method can repeat the outer loop 25 times at 1304. Afterwards, the computer-implemented method can return r at 1322.

Finally, the computer-implemented method can apply gradient descent at 1208 in FIG. 12 to refine parameters even further. Here is the gradient of the objective in some embodiments:

$$\begin{matrix}∇ F = ( \begin{matrix}\frac{\partial F}{\partial {s}_{1}} & \frac{\partial F}{\partial {s}_{2}} & \frac{\partial F}{\partial {s}_{3}} & \frac{\partial F}{\partial α} & \frac{\partial F}{\partial β} & \frac{\partial F}{\partial γ} & \frac{\partial F}{\partial {θ}_{x}} & \frac{\partial F}{\partial {θ}_{y}} & \frac{\partial F}{\partial {θ}_{z}}\end{matrix} ) \\ \frac{\partial F}{\partial {s}_{1}} = fk {θ}_{z} \\ \frac{\partial F}{\partial {s}_{2}} = gk {θ}_{z} \\ \frac{\partial F}{\partial {s}_{3}} = - ( f {\tilde{p}}_{x} + g {\tilde{p}}_{y} ) {θ}_{z} {k}^{2} \\ \frac{\partial F}{\partial α} = - ( fk {\tilde{p}}_{x} {p}_{y} + g ( {p}_{z} + {kp}_{y} {\tilde{p}}_{y} ) ) k {θ}_{z} \\ \frac{\partial F}{\partial β} = ( f ( {p}_{z} + {kp}_{x} {\tilde{p}}_{x} ) + gk {\tilde{p}}_{y} {p}_{x} ) k {θ}_{z} \\ \frac{\partial F}{\partial γ} = - ( {fp}_{y} - {gp}_{x} ) k {θ}_{z} \\ \frac{\partial F}{\partial {θ}_{x}} = - f \\ \frac{\partial F}{\partial {θ}_{y}} = - g \\ \frac{\partial F}{\partial {θ}_{z}} = k ( f {\tilde{p}}_{x} + g {\tilde{p}}_{y} )\end{matrix} \\ Where \\ \begin{matrix}\tilde{p} = Tp , k = \frac{1}{{\tilde{p}}_{z}} \\ f = {θ}_{z} k {\tilde{p}}_{x} - ( {q}_{x} + {θ}_{x} ) \\ g = {θ}_{z} k {\tilde{p}}_{y} - ( {q}_{y} + {θ}_{y} )\end{matrix}$$

The negation of the gradient gives us a direction in parameter space in which objective decreases. The magnitude of parameters delta is computed separately by the line-search algorithm. It is very similar to the iterative process mentioned in the linear refinement stage: the process shall stop if error decreases, and decrease the gradient by fixed ratio otherwise. Also momentum may or may not be used in various implementations.

In some embodiments, the modified gradient formula may be used instead. In some embodiments, if partial derivatives are simplified with respect to the α, β, γ, the convergence properties could improve. So instead of aforementioned derivatives, the following may be used:

$$\frac{\partial F}{\partial α} = - ( g + {k}^{2} {\tilde{p}}_{y} ( f {\tilde{p}}_{x} + g {\tilde{p}}_{y} ) ) {θ}_{z} \\ \frac{\partial F}{\partial β} = ( f + {k}^{2} {\tilde{p}}_{x} ( f {\tilde{p}}_{x} + g {\tilde{p}}_{y} ) ) {θ}_{z}$$

Lastly, select the best solution among the set of solutions induced by the set of focuses. That would be the final camera model. In some embodiments, this can include selecting parameters with minimal reprojection error 1212 in FIG. 12.

Number of pairs of points is between 2 and 5. In this case the full solution does not produce stable results, even though the linear system is determined when number of pairs is equal to 4, so a more limited approximation shall be used. The key differences are: absence of gradient descent step, another linear approximation with only 4 variables, namely s₁, s₂, s₃ and y.

Unlike previous case, here the best focus is not determined, which could be adjusted via control. Same applies for other parameters which are not optimize: α and β are adjusted via pointing device and θₓ, θ_{y} are assumed fixed.

Due to the absence of some parameters, the linear system becomes the following:

$$\{ \begin{matrix}{θ}_{z} {s}_{1} + 0 {s}_{2} + {q}_{x} {s}_{3} + {θ}_{z} {p}_{y} γ = 0 \\ 0 {s}_{1} - {θ}_{z} {s}_{2} + {q}_{y} {s}_{3} - {θ}_{z} {p}_{x} γ = 0 \\ …\end{matrix}$$

Similarly, by preposition it has more than 4 equations so it is (over) determined. After solving this linear system, the similar weighting algorithm should be applied to get the solution.

If there are more than 2 points given, the previous step is repeated until convergence or for a fixed number of iterations.

### Aligning 2D Digital Images

Some embodiments can include a computer-implemented method of aligning a first two dimensional (2D) digital image of at least a portion of the patient's dentition with a second 2D digital image of at least a portion of the patient's detention.

In some embodiments, the method of aligning the first and second 2D digital images can include receiving a first 2D digital image and a second 2D digital image of at least a portion of a patient's dentition.

In some embodiments, the first 2D digital image and the second 2D digital image can be digital photos taken as described previously in the disclosure. The first and second 2D digital images are of the same person in some embodiments, and can include at least a portion of a patient's mouth region, with one or more teeth exposed. In some embodiments, the first and/or second 2D digital image can be of a patient's face, including the eyes and mouth region, with one or more teeth exposed. In some embodiments, the first and second 2D digital image can be of a patient smiling, thereby exposing one or more teeth. In some embodiments, the exposed teeth can be upper jaw teeth. In some embodiments, the exposed teeth can be lower jaw teeth. In some embodiments, the exposed teeth can include at least some upper jaw teeth and some lower jaw teeth. In some embodiments, one or more upper and/or lower gum regions can also be exposed.

In some embodiments, the first and second 2D digital images can each include at least one virtual jaw with one or more virtual teeth corresponding to the one or more exposed teeth. In some embodiments, the first and second 2D digital images can each include a virtual upper jaw with one or more upper jaw virtual teeth corresponding to the one or more exposed upper jaw teeth. In some embodiments, the first and second 2D digital image can each include a virtual lower jaw with one or more lower jaw virtual teeth corresponding to the one or more exposed lower jaw teeth.

In some embodiments, the first and second 2D digital image can be the same image. In some embodiments, the first and second 2D digital image can be different images of the same person, including at least one or more virtual teeth. For example, in some embodiments, the first 2D digital image can include a mouth region with one or more virtual teeth visible as the person smiles, and the second 2D digital image can include a mouth region that has been retracted to display one or more virtual teeth as well as surrounding dentition such as a gum region and/or more. In some embodiments, the first 2D digital image can include a mouth region with one or more virtual teeth visible as the person smiles, and the second 2D digital image can include a mouth region showing one or more idealized virtual teeth. The idealized virtual teeth can be a desired look for the virtual teeth, and can be generated using photoshop or other image altering and/or editing software known in the art. This can allow, for example, a dentist to show a person a goal or final product of their dental treatment. In some embodiments, the first and/or second 2D digital images can show a full face, or portions of the face, in addition to the virtual mouth region and the virtual teeth.

For example, in some embodiments, the first 2D digital image can be an entire face including a mouth region with one or more virtual teeth visible, and the second 2D digital image can be of just the mouth region with virtual teeth visible.

In some embodiments, where the first and second 2D digital images each show the person's eyes, the two digital images can be aligned horizontally by selecting points on each pupil in one of the 2D digital images. In some embodiments, one or more key points on corresponding virtual teeth in the first and second 2D digital image can be selected.

In some embodiments, the first and/or second 2D digital image can also be mapped or aligned to a 3D digital surface model.

In some embodiments, the computer-implemented method can displaying the first 2D digital image and the second 2D digital image on a display to allow a user to select one or more key points on the first 2D digital image and one or more key points on the second 2D digital image. In some embodiments, the computer-implemented method can pair each key point selected on the first 2D digital image with a corresponding key point on the second 2D digital image. In some embodiments, pairing is performed based on the sequence of key points selected on the first and second 2D digital images.

Some embodiments can include receiving one or more key points selected on the first 2D digital image selected. In some embodiments, at least two key points selected on the first 2D digital image can be received. In some embodiments, the one or more key points selected on the first 2D digital image are on a first 2D digital image virtual tooth. In some embodiments, the one or more key points selected on the first 2D digital image selected are selected by manually by a user using an input device. In some embodiments, the computer-implemented method can receive one or more key points selected on the second 2D digital image selected. In some embodiments, the computer-implemented method can receive at least two key points selected on the second 2D digital image. In some embodiments, the one or more key points selected on the second 2D digital image are on a second 2D digital image virtual tooth. In some embodiments, the second 2D digital image virtual tooth corresponds to the first 2D digital image virtual tooth. In some embodiments, the one or more key points selected on the second 2D digital image correspond in location on the second 2D digital image to the one or more key points selected on the first 2D digital image on the first 2D digital image.

In some embodiments, the one or more key points on the first 2D digital image and the one or more corresponding key points on the second 2D digital image can be chosen automatically using a masked first 2D digital image and a masked second 2D digital image. The computer-implemented method can determine they key points automatically as discussed previously with respect to the masked 2D digital image.

In some embodiments, the computer-implemented method can perform a 2D image transform using the one or more key points selected on the first 2D digital image and the one or more key points selected on the second 2D digital image. In some embodiments, a first and second key points selected on the first 2D digital image (for example with the person's face) are designated as p_1 and p_2, respectively, and In some embodiments, and corresponding first and second key points on the second image (with the person's mouth) are designated as q_1 and q_2 (so p_2y is the Y coordinate of the second point on the first image). Then, the following system of linear equations is solved to obtain matrix T_2d, which transforms the first 2D digital image into the second 2D digital image:

$$\{ \begin{matrix}{p}_{1 x} {a}_{1} - {p}_{1 y} {a}_{2} + {a}_{3} + 0 = {q}_{1 x} \\ {p}_{1 y} {a}_{1} + {p}_{1 x} {a}_{2} + 0 + {a}_{4} = {q}_{1 y} \\ {p}_{2 x} {a}_{1} - {p}_{2 y} {a}_{3} + {a}_{3} + 0 = {q}_{2 x} \\ {p}_{2 y} {a}_{1} + {p}_{2 x} {a}_{2} + 0 + {a}_{4} = {q}_{2 y}\end{matrix} ⟹ {T}_{2 d} = ( \begin{matrix}{a}_{1} & - {a}_{2} & {a}_{3} \\ {a}_{2} & {a}_{1} & {a}_{4} \\ 0 & 0 & 1\end{matrix} )$$

In some embodiments, the system is determined (the number of variables is equal to the number of equations), so it has exactly one solution. In some embodiments, the obtained matrix is then used to transform the first 2D image so that the two uploaded images appear aligned in the scene.

In some embodiments, a final output can include placing the first 2D digital image and the second 2D digital image (or real photo with corresponding smile) relative to the camera using the final transformation matrix and displaying both on the user's display. Some embodiments can include providing an image to image opacity GUI control to adjust the opacity of the first 2D digital image relative to the second 2D digital image.

In some embodiments, fitting the camera model can occur as one or more key point pairs are selected. In some embodiments, fitting the camera model can occur after all points are selected, and a button or other GUI element initiates fitting the camera model.

Some embodiments can include defining a cutout region in a 2D digital image. In some embodiments, the cutout region can be selected by the user by selecting a closed region such as the virtual teeth region in the 2D digital image. In some embodiments, the computer-implemented method can display the cutout region as the user defines it. In some embodiments, the computer-implemented method can, after the cutout region is defined, provide a GUI element such as a virtual slider to control a cutout region opacity. In some embodiments, the cutout region opacity can allow a user to fade in/out the cutout region. This can be used, for example, where the 2D digital image is mapped to/aligned with either another 2D digital image and/or a 3D digital surface model in some embodiments. In the case of being mapped with a 3D digital surface model, the cutout region opacity control can fade in/out between the 2D digital image virtual teeth and the 3D digital surface model virtual teeth in some embodiments.

In some embodiments a person can take a digital photo that includes at least a portion of their dentition, including one or more teeth. The digital photo can be a first 2D digital image. A dentist or any other person can edit/alter the first 2D digital image to create aesthetically pleasing or idealized virtual teeth as a second 2D digital image using editing/altering software known in the art. The computer-implemented method can receive the first and second 2D digital images and perform alignment/mapping. A dentist or other user can use the computer-implemented method to can define a cutout area of the virtual teeth region in either the first or second 2D digital image in some embodiments. The dentist or other user can then use the cutout region opacity controller to show/hide the cutout virtual teeth region. This can allow, for example, a dentist to show a patient how their teeth look currently versus how their ideal teeth will look. The computer-implemented method can also map/align either the first or second 2D digital image with a 3D digital surface model of the same person's dentition. The 3D digital surface model can be altered to show how the idealized virtual teeth can be achieved. An opacity controller can fade between the 3D digital surface model and the 2D digital image to which it is mapped/aligned. In this way, a dentist or other user can visualize various combinations of a person's current virtual teeth, idealized virtual teeth, and/or the 3D digital surface model of their teeth. A dentist can also apply changes to the 3D digital surface model using standard Computer Aided Design (“CAD”) software, for example to match the idealized virtual teeth in some embodiments.

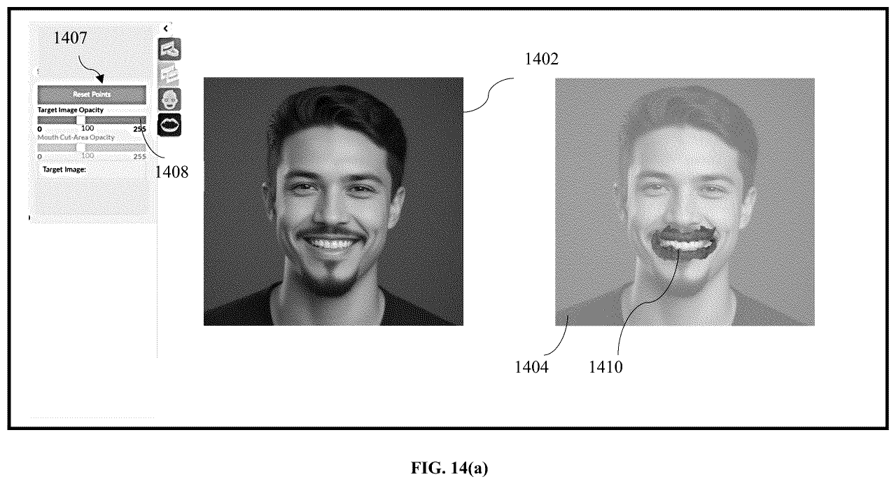

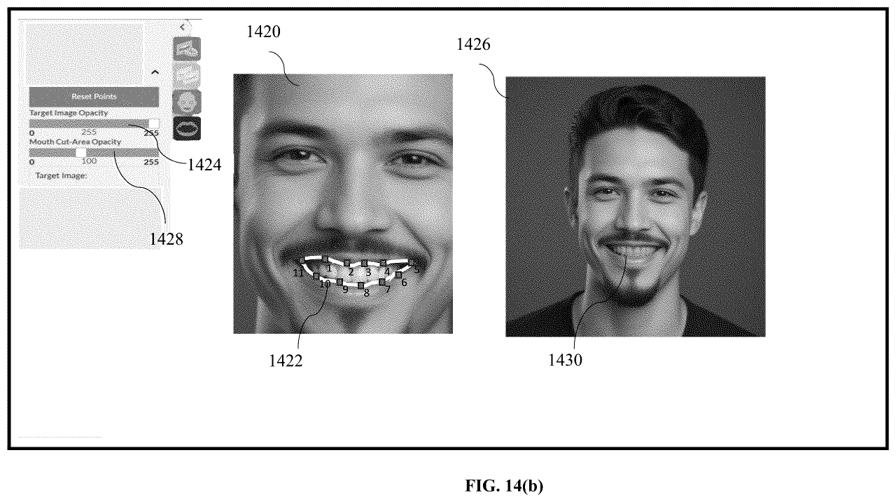

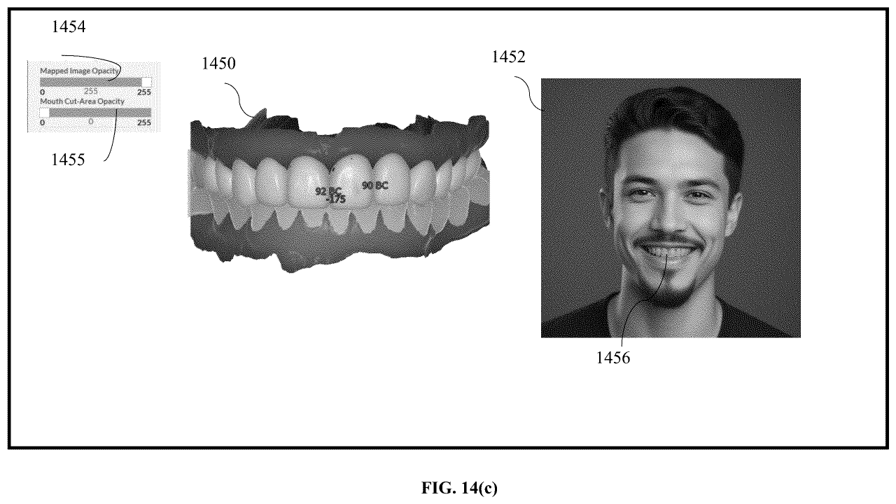

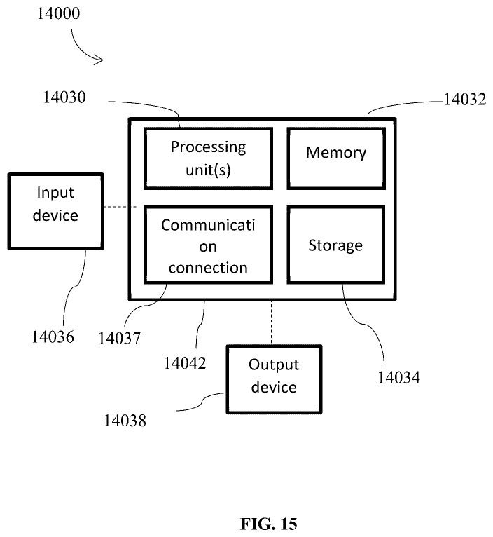

FIG. 14(a) illustrates a GUI in some embodiments. In the figure, a 2D digital image 1402 is mapped to a 3D digital surface model to provide a mapped 2D digital image 1404. A control area 1407 allows controlling the opacity of the mapped 2D digital image 1404, revealing more or less of the 3D digital surface model. For example, a target image opacity 1408 allows controlling the opacity of the mapped 2D digital image 1404. In the example of the figure, the target image opacity is set below less than the maximum opacity. Accordingly, the mapped 2D digital image 1404 is shown less opaque than a 3D digital surface model 1410 to which it is mapped. The 3D digital surface model 1410 is therefore more visible, as can be seen in the figure.

FIG. 14(b) illustrates an example in some embodiments of defining a cutout region. For example, a 2D digital image 1420 is mapped to a 3D digital surface model. Alternatively, the 2D digital image 1420 can be mapped to another 2D digital image, such as mapped 2D digital image 1426. A user-selected cutout region 1422 can define a cutout of the mouth region in the 2D digital image 1420. In the example, a target image opacity 1424 is set to maximum so that a mapped 2D digital image 1426 is fully visible. However, mouth cut-area opacity slider 1428 is set to less than the maximum value. Accordingly, the mouth cutout region is transparent, revealing a 3D digital surface model 1430 to which the mapped 2D digital image 1426 is mapped.

FIG. 14(c) illustrates an example in some embodiments of a 3D digital surface model 1450 to which a mapped 2D digital image 1452 is mapped. In the example, the mapped image opacity 1454 is set to the maximum value so that the mapped 2D digital image is fully visible. The mouth cut-area opacity 1455 is set to the minimum value, thereby revealing the 3D digital surface model 1456 in the mapped 2D digital image 1452.

Some embodiments can include a computer-implemented method of aligning at least two digital representations of at least a portion of a patient's dentition, including: receiving a two dimensional (“2D”) digital image including at least a portion of a person's dentition; receiving a three dimensional (“3D”) digital surface model of the person's dentition; receiving one or more 3D digital surface model key points selected on the 3D digital surface model; receiving one or more corresponding 2D digital image key points selected on the 2D digital image; and fitting a camera model using the one or more 3D digital surface model key points and the one or more corresponding 2D digital image key points to align the 3D digital surface model with the 2D digital image.

In some embodiments the one or more 3D digital surface model key points selected and the corresponding 2D digital image key points selected are selected manually. In some embodiments the one or more 3D digital surface model key points and the corresponding 2D digital image key points are selected automatically. In some embodiments the one or more 3D digital surface model key points selected are on one or more virtual teeth in the 3D digital surface model and the one or more corresponding 2D digital image key points selected are on one or more virtual teeth in the 2D digital image. Some embodiments can include displaying the aligned the 3D digital surface model overlaid with the aligned 2D digital image and an opacity slider to adjust visibility of the aligned 2D digital image with respect to the aligned 3D digital surface model. Some embodiments can include receiving a second 2D digital image and mapping the second 2D digital image with the 2D digital image. In some embodiments the second 2D digital image comprises a cutout region.

Some embodiments can include a system having a processor and a non-transitory computer-readable storage medium including instructions executable by the processor to perform steps including one or more features described herein, including but not limited to those described in the computer-implemented method. Some embodiments can include a non-transitory computer readable medium storing executable computer program instructions to provide aligning at least two digital representations of at least a portion of a patient's dentition, the computer program instructions having instructions for executing one or more features described herein, including but not limited to those described in the computer-implemented method.

One or more advantages of one or more features can include, for example, automatically determining an alignment between at least two digital representations of at least a portion of a patient's dentition. One or more advantages of one or more features can include, for example, a more accurate way to locate points such as using a segmented 3D digital surface model. One or more advantages of one or more features can include, for example, flexibility in the number of points, the location of key points, as well as manual or auto-selecting key points. One or more advantages of one or more features can include, for example, choosing fewer points. One or more advantages of one or more features can include, for example, fewer errors, for example. One or more advantages of one or more features can include, for example, adjustability of opacity between two or more mapped/aligned digital representations to show one or more features prominently. One or more advantages of one or more features can include, for example, allowing a user-defined cutout region. One or more advantages of one or more features can include, for example, visualizing original virtual teeth, idealized virtual teeth, and/or 3D digital surface model of virtual teeth and adjusting opacity to view and hide the virtual teeth and/or 3D digital surface model. One or more advantages can include, for example, improved accuracy and reduced error.

FIG. 15 illustrates a processing system 14000 in some embodiments. The system 14000 can include a processor 14030, computer-readable storage medium 14034 having instructions executable by the processor to perform one or more steps described in the present disclosure.

In some embodiments, one or more features can be performed by a user, for example. In some embodiments, a user using an input device such as a mouse or a finger can include one or more of the features described in the present disclosure. In some embodiments, one or more features can be performed by a user using an input device while viewing the digital model on a display, for example. In some embodiments, the computer-implemented method can allow the input device to manipulate the digital model displayed on the display. For example, in some embodiments, the computer-implemented method can rotate, zoom, move, and/or otherwise manipulate the digital model in any way as is known in the art. In some embodiments, one or more features can be performed by a user using the input device. In some embodiments, one or more features can be initiated, for example, using techniques known in the art, such as a user selecting another button.

One or more key points can be selected on one or more virtual teeth on the 2D digital image and/or the 3D digital surface model using an input device whose pointer is shown on a display, for example. The pointer can be used to select a region of one point by clicking on an input device such as a mouse or tapping on a touch screen for example. Other techniques known in the art can be used to select a point or digital surface. In some embodiments, one or more features can be performed by a user using an input device and viewing the digital model a display, for example.

In some embodiments the computer-implemented method can display a digital model on a display and receive input from an input device such as a mouse or touch screen on the display for example. For example, the computer-implemented method can receive a command to initiate mapping/alignment. The computer-implemented method can, upon receiving a mapping/alignment initiation command, perform mapping/alignment using one or more features described in the present disclosure. The computer-implemented method can, upon receiving manipulation commands, rotate, zoom, move, and/or otherwise manipulate the digital model in any way as is known in the art.

One or more of the features disclosed herein can be performed and/or attained automatically, without manual or user intervention. One or more of the features disclosed herein can be performed by a computer-implemented method. The features-including but not limited to any methods and systems-disclosed may be implemented in computing systems. For example, the computing environment 14042 used to perform these functions can be any of a variety of computing devices (e.g., desktop computer, laptop computer, server computer, tablet computer, gaming system, mobile device, programmable automation controller, video card, etc.) that can be incorporated into a computing system comprising one or more computing devices. In some embodiments, the computing system may be a cloud-based computing system.

For example, a computing environment 14042 may include one or more processing units 14030 and memory 14032. The processing units execute computer-executable instructions. A processing unit 14030 can be a central processing unit (CPU), a processor in an application-specific integrated circuit (ASIC), or any other type of processor. In some embodiments, the one or more processing units 14030 can execute multiple computer-executable instructions in parallel, for example. In a multi-processing system, multiple processing units execute computer-executable instructions to increase processing power. For example, a representative computing environment may include a central processing unit as well as a graphics processing unit or co-processing unit. The tangible memory 14032 may be volatile memory (e.g., registers, cache, RAM), non-volatile memory (e.g., ROM, EEPROM, flash memory, etc.), or some combination of the two, accessible by the processing unit(s). The memory stores software implementing one or more innovations described herein, in the form of computer-executable instructions suitable for execution by the processing unit(s).

A computing system may have additional features. For example, in some embodiments, the computing environment includes storage 14034, one or more input devices 14036, one or more output devices 14038, and one or more communication connections 14037. An interconnection mechanism such as a bus, controller, or network, interconnects the components of the computing environment. Typically, operating system software provides an operating environment for other software executing in the computing environment, and coordinates activities of the components of the computing environment.

The tangible storage 14034 may be removable or non-removable, and includes magnetic or optical media such as magnetic disks, magnetic tapes or cassettes, CD-ROMs, DVDs, or any other medium that can be used to store information in a non-transitory way and can be accessed within the computing environment. The storage 14034 stores instructions for the software implementing one or more innovations described herein.

The input device(s) may be, for example: a touch input device, such as a keyboard, mouse, pen, or trackball; a voice input device; a scanning device; any of various sensors; another device that provides input to the computing environment; or combinations thereof. For video encoding, the input device(s) may be a camera, video card, TV tuner card, or similar device that accepts video input in analog or digital form, or a CD-ROM or CD-RW that reads video samples into the computing environment. The output device(s) may be a display, printer, speaker, CD-writer, or another device that provides output from the computing environment.

The communication connection(s) enable communication over a communication medium to another computing entity. The communication medium conveys information, such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

Any of the disclosed methods can be implemented as computer-executable instructions stored on one or more computer-readable storage media 14034 (e.g., one or more optical media discs, volatile memory components (such as DRAM or SRAM), or nonvolatile memory components (such as flash memory or hard drives)) and executed on a computer (e.g., any commercially available computer, including smart phones, other mobile devices that include computing hardware, or programmable automation controllers) (e.g., the computer-executable instructions cause one or more processors of a computer system to perform the method). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media 14034. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Perl, Python, JavaScript, Adobe Flash, or any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

In view of the many possible embodiments to which the principles of the disclosure may be applied, it should be recognized that the illustrated embodiments are only examples and should not be taken as limiting the scope of the disclosure.

What is claimed is:

1. A computer-implemented method of aligning at least two digital representations of at least a portion of a patient's dentition, comprising:
receiving a two dimensional (“2D”) digital image comprising at least a portion of a person's dentition;
receiving a three dimensional (“3D”) digital surface model of the person's dentition;
receiving one or more 3D digital surface model key points selected on the 3D digital surface model;
receiving one or more corresponding 2D digital image key points selected on the 2D digital image; and
fitting a camera model using the one or more 3D digital surface model key points and the one or more corresponding 2D digital image key points to align the 3D digital surface model with the 2D digital image.

2. The method of claim 1, wherein the one or more 3D digital surface model key points selected and the corresponding 2D digital image key points selected are selected manually.

3. The method of claim 1, wherein the one or more 3D digital surface model key points and the corresponding 2D digital image key points are selected automatically.

4. The method of claim 1, wherein the one or more 3D digital surface model key points selected are on one or more virtual teeth in the 3D digital surface model and the one or more corresponding 2D digital image key points selected are on one or more virtual teeth in the 2D digital image.

5. The method of claim 1, further comprising displaying the aligned the 3D digital surface model overlaid with the aligned 2D digital image and an opacity slider to adjust visibility of the aligned 2D digital image with respect to the aligned 3D digital surface model.

6. The method of claim 1, further comprising receiving a second 2D digital image and mapping the second 2D digital image with the 2D digital image.

7. The method of claim 6, wherein the second 2D digital image comprises a cutout region.

8. A non-transitory computer readable medium storing executable computer program instructions to provide aligning at least two digital representations of at least a portion of a patient's dentition, the computer program instructions comprising instructions for:
receiving a two dimensional (“2D”) digital image comprising at least a portion of a person's dentition;
receiving a three dimensional (“3D”) digital surface model of the person's dentition;
receiving one or more 3D digital surface model key points selected on the 3D digital surface model;
receiving one or more corresponding 2D digital image key points selected on the 2D digital image; and
fitting a camera model using the one or more 3D digital surface model key points and the one or more corresponding 2D digital image key points to align the 3D digital surface model with the 2D digital image.

9. The medium of claim 8, wherein the one or more 3D digital surface model key points selected and the corresponding 2D digital image key points selected are selected manually.

10. The medium of claim 8, wherein the one or more 3D digital surface model key points and the corresponding 2D digital image key points are selected automatically.

11. The medium of claim 8, wherein the one or more 3D digital surface model key points selected are on one or more virtual teeth in the 3D digital surface model and the one or more corresponding 2D digital image key points selected are on one or more virtual teeth in the 2D digital image.

12. The medium of claim 8, further comprising displaying the aligned the 3D digital surface model overlaid with the aligned 2D digital image and an opacity slider to adjust visibility of the aligned 2D digital image with respect to the aligned 3D digital surface model.

13. The medium of claim 8, further comprising receiving a second 2D digital image and mapping the second 2D digital image with the 2D digital image.

14. The medium of claim 13, wherein the second 2D digital image comprises a cutout region.

15. A system for aligning at least two digital representations of at least a portion of a patient's dentition, the system comprising:
a processor; and
a non-transitory computer-readable storage medium comprising instructions executable by the processor to perform steps comprising:
receiving a two dimensional (“2D”) digital image comprising at least a portion of a person's dentition;
receiving a three dimensional (“3D”) digital surface model of the person's dentition;
receiving one or more 3D digital surface model key points selected on the 3D digital surface model;
receiving one or more corresponding 2D digital image key points selected on the 2D digital image; and
fitting a camera model using the one or more 3D digital surface model key points and the one or more corresponding 2D digital image key points to align the 3D digital surface model with the 2D digital image.

16. The system of claim 15, wherein the one or more 3D digital surface model key points selected and the corresponding 2D digital image key points selected are selected manually.

17. The system of claim 15, wherein the one or more 3D digital surface model key points and the corresponding 2D digital image key points are selected automatically.

18. The system of claim 15, further comprising displaying the aligned the 3D digital surface model overlaid with the aligned 2D digital image and an opacity slider to adjust visibility of the aligned 2D digital image with respect to the aligned 3D digital surface model.

19. The system of claim 15, further comprising receiving a second 2D digital image and mapping the second 2D digital image with the 2D digital image.

20. The system of claim 19, wherein the second 2D digital image comprises a cutout region.
